(19) United States
(12) Patent Application Publication
(10) Pub. No.: US 2020/0015979 A1
(43) Pub. Date: Jan. 16, 2020

(54) INTERVERTEBRAL PROSTHETIC DISC WITH SHOCK ABSORPTION CORE

(71) Applicant: Simplify Medical Pty Ltd, Paddington NSW (AU)

(72) Inventor: Yves ARRAMON, Sunnyvale, CA (US)

(73) Assignee: Simplify Medical Pty Ltd, Paddington NSW (AU)

(21) Appl. No.: 16/560,760

(22) Filed: Sep. 4, 2019

Related U.S. Application Data
(63) Continuation of application No. 15/283,728, filed on Oct. 3, 2016, now abandoned.
Application No. 15/283,728 is a continuation of application No. 14/715,900, filed on May 19, 2015, now abandoned.
Application No. 14/715,900 is a continuation of application No. 12/207,635, filed on Sep. 10, 2008, now abandoned.
(60) Provisional application No. 60/973,003, filed on Sep. 17, 2007.

(51) Int. Cl. A61F 2/44

(52) U.S. Cl. CPC A61F 2/4425; A61F 2002/30014

(57) ABSTRACT

An artificial intervertebral disc with shock absorption includes upper and lower plates disposed about a shock absorbing movable core. The upper and lower plates have an outer surface which engages a vertebrae and an inner bearing surface. The shock absorbing core includes a unitary member of a rigid material having at least one lateral cut between upper and lower surfaces of the core to allow the upper and lower surfaces to move resiliently toward and away from each other. This allows the core to absorb forces applied to it by the vertebrae.

### CROSS-REFERENCES TO RELATED APPLICATIONS

The present application is a continuation of U.S. patent application Ser. No. 15/283,728 (Attorney Docket No. 29850-718.302), filed Oct. 3, 2016, which is a continuation of U.S. patent application Ser. No. 14/715,900 (Attorney Docket No. 29850-718.301), filed May 19, 2015, which is a continuation of U.S. patent application Ser. No. 12/207,635 (Attorney Docket No. 29850-718.201), filed Sep. 10, 2008, which application claims the benefit of U.S. Provisional Application No. 60/973,003 (Attorney Docket No. 29850-718.101), filed Sep. 17, 2007; all of which are incorporated herein by reference in their entirety.

### BACKGROUND OF THE INVENTION

The present invention relates to medical devices and methods. More specifically, the invention relates to intervertebral disc prostheses.

Back pain takes an enormous toll on the health and productivity of people around the world. According to the American Academy of Orthopedic Surgeons, approximately 80 percent of Americans will experience back pain at some time in their life. In the year 2000, approximately 26 million visits were made to physicians' offices due to back problems in the United States. On any one day, it is estimated that 5% of the working population in America is disabled by back pain.

One common cause of back pain is injury, degeneration and/or dysfunction of one or more intervertebral discs. Intervertebral discs are the soft tissue structures located between each of the thirty-three vertebral bones that make up the vertebral (spinal) column. Essentially, the discs allow the vertebrae to move relative to one another. The vertebral column and discs are vital anatomical structures, in that they form a central axis that supports the head and torso, allow for movement of the back, and protect the spinal cord, which passes through the vertebrae in proximity to the discs.

Discs often become damaged due to wear and tear or acute injury. For example, discs may bulge (herniate), tear, rupture, degenerate or the like. A bulging disc may press against the spinal cord or a nerve exiting the spinal cord, causing “radicular” pain (pain in one or more extremities caused by impingement of a nerve root). Degeneration or other damage to a disc may cause a loss of “disc height,” meaning that the natural space between two vertebrae decreases. Decreased disc height may cause a disc to bulge, facet loads to increase, two vertebrae to rub together in an unnatural way and/or increased pressure on certain parts of the vertebrae and/or nerve roots, thus causing pain. In general, chronic and acute damage to intervertebral discs is a common source of back related pain and loss of mobility.

When one or more damaged intervertebral discs cause a patient pain and discomfort, surgery is often required. Traditionally, surgical procedures for treating intervertebral discs have involved discectomy (partial or total removal of a disc), with or without fusion of the two vertebrae adjacent to the disc. Fusion of the two vertebrae is achieved by inserting bone graft material between the two vertebrae such that the two vertebrae and the graft material grow together. Oftentimes, pins, rods, screws, cages and/or the like are inserted between the vertebrae to act as support structures to hold the vertebrae and graft material in place while they permanently fuse together. Although fusion often treats the back pain, it reduces the patient's ability to move, because the back cannot bend or twist at the fused area. In addition, fusion increases stresses at adjacent levels of the spine, potentially accelerating degeneration of these discs.

In an attempt to treat disc related pain without reducing intervertebral mobility, an alternative approach to fusion has been developed, in which a movable, implantable, artificial intervertebral disc (or “disc prosthesis”) is inserted between two vertebrae. A number of different artificial intervertebral discs are currently being developed. For example, U.S. Patent Application Publication Nos. 2005/0021146, 2005/0021145, and 2006/0025862, which are hereby incorporated by reference in their entirety, describe artificial intervertebral discs. Other examples of intervertebral disc prostheses are the LINK SB Charite™ disc prosthesis (provided by DePuy Spine, Inc.), the MOBIDISK™ disc prosthesis (provided by LDR Medical), the BRYAN™ cervical disc prosthesis (provided by Medtronic Sofamor Danek, Inc.), the PRODISC™ disc prosthesis, or PRODISC-C™ disc prosthesis (from Synthes Stratec, Inc.), and the PCM™ disc prosthesis (provided by Cervitech, Inc.). Although existing disc prostheses provide advantages over traditional treatment methods, improvements are ongoing.

The known artificial intervertebral discs generally include upper and lower plates or shells which locate against and engage the adjacent vertebral bodies, and a core for providing motion between the plates. The core may be movable or fixed, metallic or polymer and generally has at least one convex outer surface which mates with a concave recess on one of the plate in a fixed core device or both of the plates for a movable core device such as described in U.S. Patent Application Publication No. 2006/0025862. However, currently available artificial intervertebral discs do not provide for cushioning or shock absorption which would help absorb forces applied to the prosthesis from the vertebrae to which they are attached. A natural disc is largely fluid which compresses to provide cushioning. It would be desirable to mimic some of this cushioning in an artificial disc.

De Villiers et al., US 2006/0178766 A1 “Intervertebral prosthetic disc with shock absorption”, the entirety of which is hereby incorporated by reference, describes a mobile core with an elastic component sandwiched between hardened spherical surfaces.

Therefore, a need exists for improved artificial intervertebral disc. Ideally, such improved disc would avoid at least some of the short comings of the present discs while provided shock absorption.

### BRIEF SUMMARY OF THE INVENTION

Embodiments of the present invention provide an artificial intervertebral disc with shock absorption and methods of providing shock absorption with an artificial disc. The prosthesis system comprises supports that can be positioned against vertebrae and a shock absorbing core that can be positioned between the supports to allow the supports to articulate.

In a first aspect, embodiments of the present invention provide an artificial intervertebral disc. The artificial intervertebral disc comprises upper and lower supports. Each support comprises an outer surface which engages a vertebra and an inner bearing surface. A core comprises upper and lower surfaces. The upper and lower surfaces of the core are configured to engage the inner bearing surfaces of the upper and lower support plates. The core is formed as a unitary member with at least one lateral cut positioned between the upper and lower surfaces to allow the upper and lower surfaces of the core to move resiliently toward and away from each other.

In another aspect, embodiments of the present invention provide a method of implanting an artificial intervertebral disc in an intervertebral space. Upper and lower supports are provided, in which each support comprises an outer surface that engages a vertebra and an inner surface. A core is provided that comprises upper and lower surfaces that engage the inner surfaces of the upper and lower supports. The core comprises at least one lateral cut disposed between the upper and lower surfaces. The core and the supports are inserted into the intervertebral space such at least one uncut portion of the core resiliently flexes and urges the upper and lower surfaces of the core away from each other when the core is inserted into the intervertebral space.

### BRIEF DESCRIPTION OF THE DRAWINGS

FIG. 1A is a side cross sectional view of an artificial disc with a shock absorption core;

FIG. 1B shows a side view of the prosthetic disc in FIG. 1A after sliding movement of the plates over the core;

FIG. 2 is a side view of the shock absorbing core of FIG. 1A, according to one embodiment of the present invention;

FIG. 3 is a top cross sectional view of the shock absorbing core of FIG. 1A having four cuts;

FIG. 4 is a cross sectional view of another example of the shock absorbing core of FIG. 1A having five or more cuts;

FIG. 5 is a cross sectional view of another example of the shock absorbing core of FIG. 1A having four or more cuts;

FIG. 6 is a cross sectional view of another example of the shock absorbing core of FIG. 1A having six or more cuts;

FIG. 7 is a cross sectional view of another example of the shock absorbing core of FIG. 1A having three or more shallow cuts;

FIG. 8 is a cross sectional view of another example of the shock absorbing core of FIG. 1A having four or more shallow cuts;

FIG. 9 is a cross sectional view of another example of the shock absorbing core of FIG. 1A having multiple cuts of varying depths providing one or more preferential deflection direction;

FIG. 10 is a side view of a shock absorbing core according to one embodiment of the present invention with tapered cuts to provide a non-linear spring stiffness;

FIG. 11 is a perspective view of the core of FIG.10;

FIG. 12 is a side view of a shock absorbing core according to another embodiment of the present invention with a spiral cut; and

FIG. 13 is a perspective view of the core of FIG. 12.

### DETAILED DESCRIPTION OF THE INVENTION

Various embodiments of the present invention generally provide for an artificial intervertebral disc having upper and lower plates disposed about a shock absorbing movable core. The shock absorbing core includes a rigid material having at least one lateral cut between upper and lower surfaces of the core to allow the upper and lower surfaces to move resiliently toward and away from each other. This allows the core to absorb forces applied to it by the vertebrae. The shock absorbing cores described herein can be used with many artificial disc designs and with different approaches to the intervertebral disc space including anterior, lateral, posterior and posterior lateral approaches. Although various embodiments of such an artificial disc are shown in the figures and described further below, the general principles of these embodiments, namely providing a resilient unitary core with a force absorbing design, may be applied to any of a number of other disc prostheses, such as but not limited to the LINK SB Chariteim disc prosthesis (provided by DePuy Spine, Inc.) MOBIDISK™ disc prosthesis (provided by LDR Medical), the BRYAN™ cervical disc prosthesis, and Maverick Lumbar Disc (provided by Medtronic Sofamor Danek, Inc.), the PRODISC™ or PRODISC-™ (from Synthes Stratec, Inc.), and the PCM™ disc prosthesis (provided by Cervitech, Inc.). In some embodiments, the shock absorbing core can be used with an expandable intervertebral prosthesis, as described in U.S. Publication No. US 2007/0282449, entitled “Posterior Spinal Device and Method”, filed Apr. 12, 2007, the full disclosure of which is incorporated herein by reference.

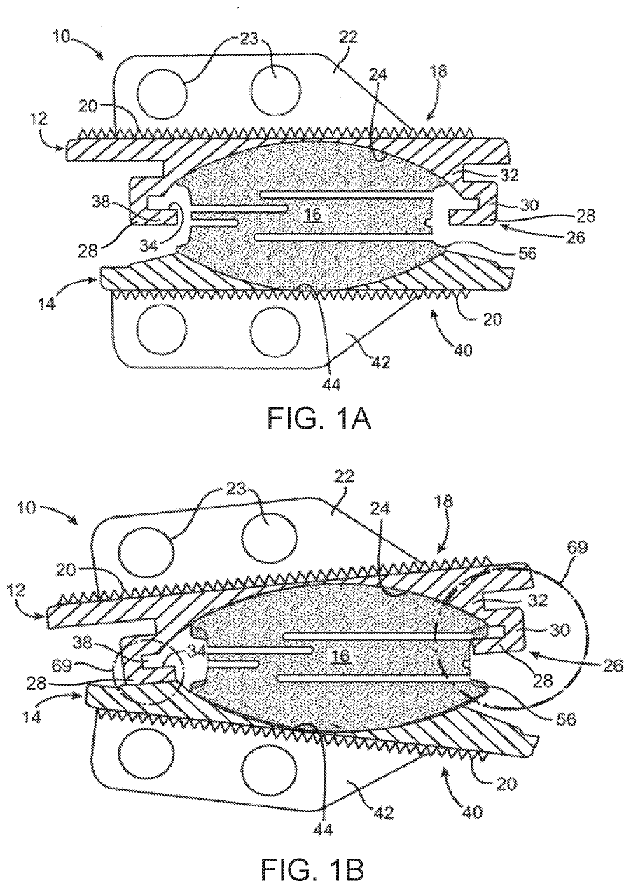

FIGS. 1A and 1B show an artificial disc 10 having a shock absorbing core 16, according to embodiments of the present invention. FIG. 1B shows a side view of the prosthetic disc after sliding movement of the plates over the core. Disc 10 for intervertebral insertion between two adjacent spinal vertebrae (not shown) includes an upper plate 12, a lower plate 14 and a movable shock absorbing core 16 located between the plates. The upper plate 12 includes an outer surface 18 and an inner surface 24 and may be constructed from any suitable material including metal, alloy, ceramic, polymer or combination of materials, such as but not limited to cobalt chrome molybdenum alloys, titanium (such as grade 5 titanium), stainless steel, reinforced ceramic, PEEK, or reinforced PEEK and combinations thereof. In one embodiment, typically used in the lumbar spine, the upper plate 12 is constructed of cobalt chrome molybdenum, and the outer surface 18 is treated with aluminum oxide blasting followed by a titanium plasma spray. In another embodiment, typically used in the cervical spine, the upper plate 12 is constructed of titanium, the inner surface 24 is coated with titanium nitride, and the outer surface 18 is treated with aluminum oxide blasting. An alternative cervical spine embodiment includes no coating on the inner surface 24. In other cervical and lumbar disc embodiments, any other suitable metals or combinations of metals may be used. In some embodiments, it may be useful to couple two materials together to form the inner surface 24 and the outer surface 18. For example, the upper plate 12 may be made of an MRI-compatible material, such as titanium, but may include a harder material, such as cobalt chrome molybdenum, for the inner surface 24. In another embodiment, upper plate 12 may comprise a metal layer or screen for improved bone integration, and inner surface 24 may comprise a PEEK or ceramic material for better imaging. All combinations of materials are contemplated within the scope of the present invention. Any suitable technique may be used to couple materials together, such as snap fitting, slip fitting, lamination, interference fitting, use of adhesives, welding and/or the like. Any other suitable combination of materials and coatings may be employed in various embodiments of the invention.

In some embodiments, the outer surface 18 is planar. Oftentimes, the outer surface 18 will include one or more surface features and/or materials to enhance attachment of the prosthesis 10 to vertebral bone. For example, the outer surface 18 may be machined to have serrations 20 or other surface features for promoting adhesion of the upper plate 12 to a vertebra. In the embodiment shown, the serrations 20 extend in mutually orthogonal directions, but other geometries would also be useful. Additionally, the outer surface 18 may be provided with a rough microfinish formed by blasting with aluminum oxide microparticles or the like. In some embodiments, the outer surface may also be titanium plasma sprayed to further enhance attachment of the outer surface 18 to vertebral bone.

The outer surface 18 may also carry one or more upstanding, vertical fins 22 extending in an anterior-posterior direction. In one embodiment, the fin 22 is pierced by transverse holes 23 for bone ingrowth. In alternative embodiments, the fin 22 may be rotated away from the anterior-posterior axis, such as in a lateral-lateral orientation, a posterolateral-anterolateral orientation, or the like. In some embodiments, the fin 22 may extend from the surface 18 at an angle other than 90°. Furthermore, multiple fins 22 may be attached to the surface 18 and/or the fin 22 may have any other suitable configuration, in various embodiments. In some embodiments, such as discs 10 for cervical insertion, the fins 22, 42 may be omitted altogether.

The inner, spherically curved concave surface 24 provides a bearing surface for the shock absorbing core 16. At the outer edge of the curved surface 24, the upper plate 12 carries peripheral restraining structure comprising an integral ring structure 26 including an inwardly directed rib or flange 28. The flange 28 forms part of a U-shaped member 30 joined to the major part of the plate by an annular web 32.

The lower plate 14 is similar to the upper plate 12 except for the absence of the peripheral restraining structure 26. Thus, the lower plate 14 has an outer surface 40 which is planar, serrated and microfinished like the outer surface 18 of the upper plate 12. The lower plate 14 optionally carries one or more fins 42 similar to the fin 22 of the upper plate. The inner surface 44 of the lower plate 14 is concavely, spherically curved with a radius of curvature matching that of the shock absorbing core 16 to provide a bearing surface for the core. Once again, the inner surface 44 may be provided with a titanium nitride or other finish.

At the outer edge of the inner curved surface 44, the lower plate 14 is provided with an inclined ledge formation 46 which contacts the flange 28 of the upper plate to limit the range of motion of the plates. Alternatively, the lower plate 14 may include peripheral restraining structure analogous to the peripheral restraining structure 26 on the upper plate 12. The peripheral restraining structure 26 may be omitted from the upper plate 12 when another retaining structure is present on the lower plate 14.

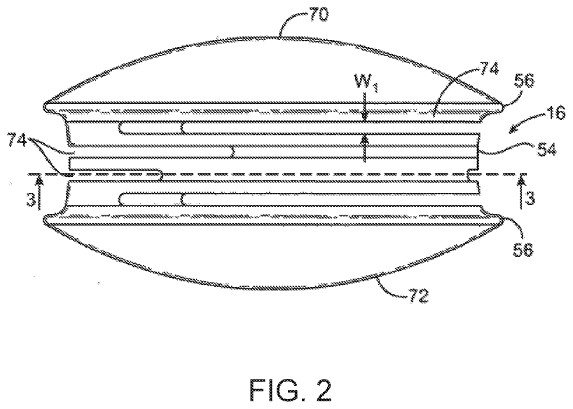

The shock absorbing core 16 shown in FIG. 2 and described herein has an exterior shape which is symmetrical about a central, equatorial plane. Although in other embodiments, the shock absorbing core 16 may be asymmetrical. Lying on this equatorial plane is an annular recess or groove 54 which extends about the periphery of the shock absorbing core. The groove 54 is defined between upper and lower ribs or lips 56. When the plates 12, 14 and shock absorbing core 16 are assembled and in the orientation seen in FIG. 1A, the flange 28 on the upper plate 12 is aligned with the groove 54 of the core so that as the core moves it is retained by engagement of the flange 28 into the groove. The flange 28 and the groove 54 defined between the ribs 56, prevent separation of the core from the plates. In other words, the cooperation of the retaining formations ensures that the shock absorbing core is held captive between the plates at all times during flexure of the disc 10.

The outer diameter of the lips 56 is preferably very slightly smaller than the diameter defined by the inner edge of the flange 28 to allow the core to be placed into the opening in the top plate 12. In another embodiment, the shock absorbing core 16 is fitted into the upper plate 12 via an interference fit. To form such an interference fit with a metal component of selected core 16 and metal plate 12, any suitable techniques may be used. For example, the plate 12 may be heated so that it expands, and the core 16 may be dropped into the plate 12 in the expanded state. When the plate 12 cools and contracts the interference fit is created. In another embodiment, the upper plate 12 may be formed around the component of shock absorbing core 16. Alternatively, the shock absorbing core 16 and upper plate 12 may include complementary threads, which allow the selected shock absorbing core 16 to be screwed into the upper plate 12, where it can then freely move.

In an alternative embodiment, the continuous annular flange 28 may be replaced by a retaining formation comprising a number of flange segments which are spaced apart circumferentially. Such an embodiment could include a single, continuous groove 54 as in the illustrated embodiment. Alternatively, a corresponding number of groove-like recesses spaced apart around the periphery of the selected core could be used, with each flange segment opposing one of the recesses. In another embodiment, the continuous flange or the plurality of flange segments could be replaced by inwardly directed pegs or pins carried by the upper plate 12. This embodiment could include a single, continuous groove 54 or a series of circumferentially spaced recesses with each pin or peg opposing a recess. Alternately, the retention feature can include one or more pegs or pins formed on the core while a corresponding groove or channel for engaging the pegs if formed in one of the plates.

In yet another embodiment, the retaining formation(s) can be carried by the lower plate 14 instead of the upper plate, i.e. the plates are reversed. In some embodiments, the upper (or lower) plate is formed with an inwardly facing groove, or circumferentially spaced groove segments, at the edge of its inner, curved surface, and the outer periphery of the selected core is formed with an outwardly facing flange or with circumferentially spaced flange segments.

In use, the disc 10 is surgically implanted between adjacent spinal vertebrae in place of a damaged disc. The adjacent vertebrae arc forcibly separated from one another to provide the necessary space for insertion. The disc 10 is typically, though not necessarily, advanced toward the disc space from an anterolateral or anterior approach and is inserted in a posterior direction—i.e., from anterior to posterior. The disc 10 is inserted into place between the vertebrae with the fins 22, 42 of the top and bottom plates 12, 14 entering slots cut in the opposing vertebral surfaces to receive them. During and/or after insertion, the vertebrae, facets, adjacent ligaments and soft tissues are allowed to move together to hold the disc in place. The serrated and microfinished surfaces 18, 40 of the plates 12, 14 locate against the opposing vertebrae. The serrations 20 and fins 22, 42 provide initial stability and fixation for the disc 10. With passage of time, enhanced by the titanium surface coating, firm connection between the plates and the vertebrae will be achieved as bone tissue grows over the serrated surface. Bone tissue growth will also take place about the fins 22, 40 and through the transverse holes 23 therein, further enhancing the connection which is achieved.

In the assembled disc 10, the complementary and cooperating spherical surfaces of the plates 12, 14 and shock absorbing core 16 allow the plates to slide or articulate over the core through a fairly large range of angles and in all directions or degrees of freedom, including rotation about the central axis. FIG. 1A shows the disc 10 with the plates 12 and 14 and shock absorbing core 16 aligned vertically with one another.

Referring now to FIG. 2, a side view of a shock absorbing core 16 is shown in detail. The core 16 includes an upper convex surface 70 and a lower convex surface 72. The core 16 is formed as a unitary member with at least one lateral cut 74 positioned between the upper and lower surfaces 70, 72 to allow the upper and lower surfaces of the core to move resiliently toward and away from each other. The unitary or one piece construction of the shock absorbing core 16 provides significant advantages over multi-part cores both in durability and manufacturability. The lateral cuts 74 in the core allow the core to function as a compliant member without affecting the function of the upper and lower convex articulating surfaces of the core 70, 72.

Preferably, the core 16 is made of biocompatible metal such as titanium, cobalt chromium alloy, stainless steel, tantalum, PEEK, or a combination thereof In addition, “superelastic” materials may be employed to leverage tolerance to large strains (e.g. NiTi alloy, or “Nitinol”). These materials provide a high hardness surface for the upper and lower surfaces 70, 72 which improve performance and prevent particulate generation. These materials also can be designed to provide a device which is deformable in the elastic region of the stress/strain curve and will not plastically deform during compression.

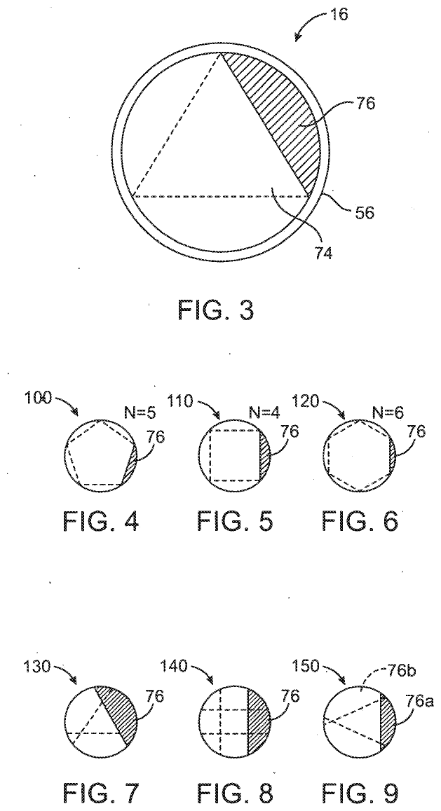

FIG. 3 shows a cross-section through the core 16 taken along the line 3-3 of FIG. 2. The lateral cuts or slits 74 in the embodiment of FIGS. 2 and 3 extend into the core in three different directions which are each 120 degrees from each other. The number of cuts can be varied to change the amount of compliance of the core 16. However, four cuts 74 in three directions have been illustrated. When a load is applied to the upper and lower surfaces 70, 72 of the core 16 the core will compress with each of the cuts 74 closing and the total amount of compression possible depending on the number, arrangement, and height of the cuts. In the embodiment of FIGS. 2 and 3, the cuts 74 form cantilevered portions above and below each of the cuts which function like cantilevers or leaf springs to allow the core to be compressed.

FIGS. 4, 5 and 6 show cores 100, 110, and 120 with different numbers of cutting directions. There may be one or more than one cut in each of the cutting directions. The material remaining after the cuts 74 are made in the cores is called a column 76. A shallow cut 74 and a large column 76 provides a stiffer core, while a deeper cut and smaller column provides a more compliant core. In the embodiments shown in FIGS. 1-6 the cuts are at least two thirds of the way through the core width or diameter, and preferably at least three quarters of the way through the core width.

FIGS. 7 and 8 show cores 130 and 140 with shallower cuts and larger columns 76. In the embodiments shown in FIGS. 7 and 8 the cuts are at least one half of the way through the core width or diameter, and less than three quarters of the way through the core width. These core designs can provide more stability in shear while compliance can be increased by increasing cut thickness or number of cuts.

FIG. 9 shows and alternative embodiment of a shock absorbing core 150 which preferential deflection in one or more bending directions. Preferential deflection is useful in combination with a directional core which is either fixed to the upper or lower plate 12, 14 or has limited ability to rotate within the upper and lower plates. In one example, the preferential deflection shock absorbing core 150 can have one small column 76a and two larger columns 76b . For example, for higher compliance in the anterior direction, the small column 76a is located in on the posterior side. Alternately, preferential compliance can be provided in two opposite directions, i.e. posterior and anterior by providing two small columns on posterior and anterior sides and larger columns on the lateral sides.

FIGS. 1-9 illustrate embodiments of the shock absorbing core with lateral cuts in multiple directions with the lateral cuts each having a slot width Wi in FIG. 2 which is substantially constant along the cuts. This constant width of the cuts provides a device which has a hard stop. However, the lateral cuts can also be designed with varying widths to tailor the compliance properties of the core.

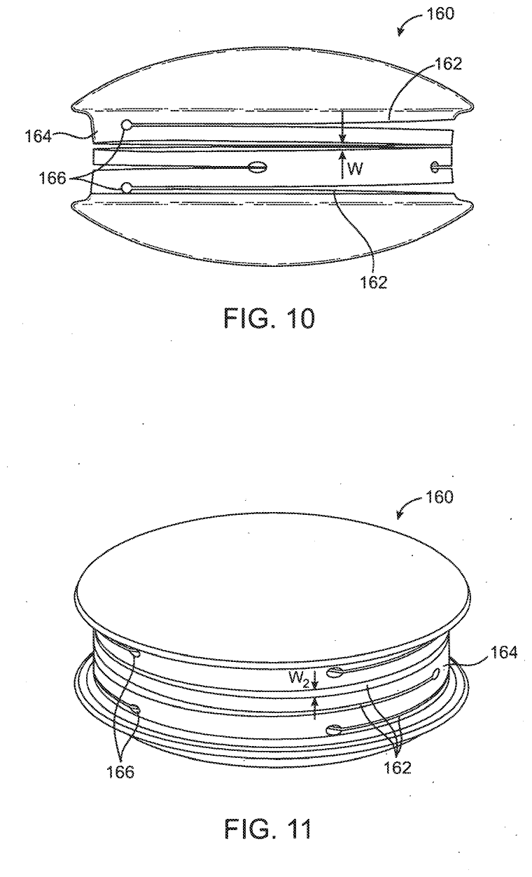

FIGS. 10 and 11 illustrate a variable stiffness shock absorbing core 160 having cuts 162 with tapering widths W2. The width of the cuts 162 is smallest where the cut terminates adjacent the column 164 and is largest at the edge of the core furthest from the column. In this version, each of the cuts 162 acts as a non linear spring providing progressively stiffer behavior upon larger compression. This is due to the fact that progressively more material on the sides of the cuts 162 is in contact as the core 160 is compressed. The non-linear spring can be incorporated in any of the other embodiments described herein to provide a softer stop to the compliant action of the core. The tapered width cuts 162 can provide the additional benefit of providing a flushing action during operation that moves any accumulated material out of the cuts.

As shown in FIGS. 10 and 11, the cuts 162 also include a stress relief 166 at the end of the cuts 162 which increases the fatigue life of the device by reducing the stress concentration at the ends of the cuts. These stress relief 166 can be provided in any of the embodiments described herein.

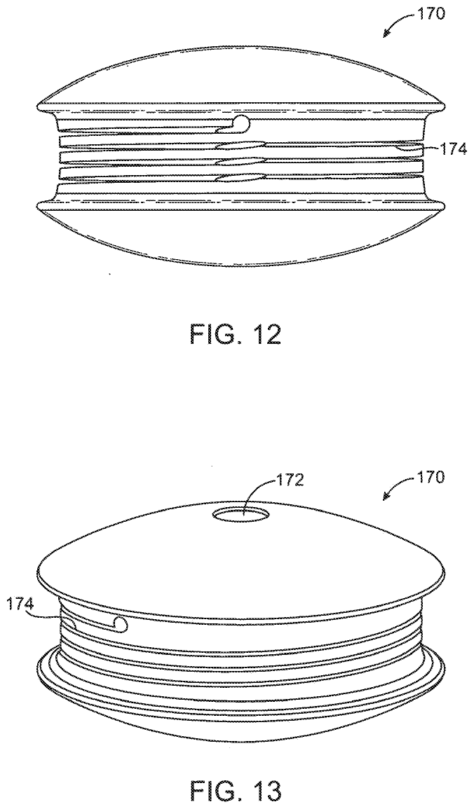

An alternative embodiment of a shock absorbing core 170 is illustrated in FIGS. 12 and 13. The core 170 includes a central bore 172 and a spiral cut 174. The spiral cut 174 intersects the central bore 172 and forms a continuous spring element which provides compliance to the core. Although the spiral cut core 170 is illustrated with one spiral cut, multiple spiral cuts may also be employed. For example, two or more spiral cuts arranged in opposite directions can be formed in the core. The compression of a spiral cut core 170 can result in some small amount of relative rotation between the upper and lower surfaces 70, 72. In cases where it is desirable to eliminate this rotation, a core having multiple spiral cuts in opposite directions can be used. For example, a core can be formed with a first spiral cut at a top of the core in a first direction and a second spiral cut at a bottom of the core in an opposite second direction. The first and second spiral cuts can offset rotation of each other resulting in a non rotating compliant core. The double spiral embodiment of the core is also more stable than the single coil in shear.

In each of the shock absorbing cores described herein, the interconnected sections within the cores arc designed for minimal or no motion between contacting parts to prevent particulate generation. However, since the cores are made entirely of hard materials such as metals, some minimal rubbing contact may be accommodated.

According to embodiments of the invention, the shock absorbing core 16 according to any of the embodiments described herein is manufactured by wire EDM (electrical discharge machining), molding, laser cutting, machining, grinding, diamond sawing, or the like. A number of lateral cuts 74 can vary from 1 to about 8 for a core in a cervical disc having a total core height of about 5 mm and from 1 to about 16 for a core in a lumbar disc having a total core height of about 10 mm. In most cases where spiral cuts are not used, the core will include at least 3 lateral cuts 74.

When implanted between vertebrae, the shock absorbing cores 16, 100, 110, 120, 130, 140, 150, 160 can resiliently absorb shocks transmitted vertically between upper and lower vertebrae of the patient's spinal column. This shock absorption is related to the material properties, design, and dimensions of the core. In general, an increased number and width of the cuts 74 will increase absorbance of shocks, with more elastic, or springy compression between the vertebrae.

In one embodiment of the present invention, for a cervical application, the maximum deformation of the shock absorbing disc is about 0.1 to about 1 mm, and is preferably about 0.2 to about 0.8 mm. For a lumbar application, the maximum deformation of the shock absorbing disc is about 0.1 to about 2.0 mm, and is preferably about 0.4 to about 1.2 mm. In some embodiments, the core has a minimum compression of about 0.01 mm.

The shock absorbing cores can be provided with differing heights and differing resiliencies, for different patients or applications. The cores can be designed with a maximum angle of inflection when loaded of about 10 degrees, preferably about 6 degrees. The core is relatively stiff with a stiffness varying depending on the location in the spine. In one example of a cervical disc, the stiffness of the core between the upper and lower surfaces is about 300 N/mm to about 2 MN/mm, preferably about 600-1500 N/mm. In another example a core for a lumbar disc has a stiffness between the upper and lower surfaces of about 600 N/mm to about 4 MN/mm, preferably about 1-3 MN/mm.

Although the shock absorbing core has been illustrated with respect to a movable core design of an artificial disc, the shock absorbing core can also be incorporated into one of the parts of a two piece ball and socket motion artificial disc. In the case of a ball and socket design the shock absorbing core can be incorporated into the ball or the socket portion of the artificial disc.

In many embodiments, the shock absorbing core can be compressed with an instrument during insertion to allow for a lower profile during insertion.

While the exemplary embodiments have been described in some detail, by way of example and for clarity of understanding, those of skill in the art will recognize that a variety of modifications, adaptations, and changes may be employed. Hence, the scope of the present invention should be limited solely by the appended claims.

What is claimed is:

1. An artificial intervertebral disc system comprising:
an artificial intervertebral disc comprising:
upper and lower supports, each support comprising an outer surface configured to engage a vertebra, and an inner bearing surface; and
a core comprising upper and lower surfaces configured to engage and articulate with respect to the inner bearing surfaces of the upper and lower supports,
wherein the core is formed as a unitary member with upper and lower lips formed at the upper and lower surfaces, and at least three lateral cuts positioned between the upper and lower lips to allow the upper and lower surfaces of the core to move resiliently toward and away from each other without affecting articulation of the upper and lower surfaces of the core with respect to the inner bearing surfaces of the upper and lower supports, wherein the at least three lateral cuts extend into the core to a depth of at least two thirds of a width of the core; and
an instrument configured to hold the core compressed during insertion of the artificial intervertebral disc into a patient.

2. The system of claim 1, wherein the at least three lateral cuts of the core are evenly spaced between the upper and lower lips.

3. The system of claim 1, wherein the at least three lateral cuts overlay each other in a vertical plane.

4. The system of claim 1, wherein the at least three lateral cuts extend into the core to a depth of at least one half and less than three quarters of a width of the core.

5. The system of claim 1, wherein the at least three lateral cuts are formed in a staggered arrangement with the cuts substantially evenly spaced around a periphery of the core.

6. The system of claim 1, wherein the inner bearing surface of the upper support comprises a curved surface and the upper surface of the core comprises a curved surface to slide against the inner bearing surface of the upper support.

7. The system of claim 1, wherein the lower surface of the core is capable of attachment to the lower support plate.

8. The system of claim 1, wherein the lower surface of the core slides against the inner bearing surface of the lower support when the disc is in an implanted configuration.

9. The system of claim 1, wherein the at least three lateral cuts divide the core into portions, and there is no sliding contact between the portions as the upper and lower surfaces of the core move with respect to one another in response to loading.

10. The system of claim 1, wherein the at least three lateral cuts are of uneven depth to create a core with a preferential deflection direction.

11. The system of claim 1, wherein the at least three lateral cuts have tapering cross sections to provide increasing stiffness with progressive compression.

12. The system of claim 1, wherein the core has a maximum compression of about 1 mm or less.

13. The system of claim 1, wherein the core has a minimum compression of about 0.01 mm.

14. The system of claim 1, wherein the core has a maximum angle of inflection when loaded between the upper and lower surfaces of the core of about 6 degrees.

15. The system of claim 1, wherein the core is configured for a cervical application and has a stiffness of about 300 to about 2000 N/mm between the upper and lower surfaces of the core.

16. The system of claim 1, wherein the core is configured for a lumbar application and has a stiffness of about 600 to about 2000 N/mm between the upper and lower surfaces of the core.

17. A method of implanting an artificial intervertebral disc in an intervertebral space, the method comprising:
providing an artificial intervertebral disc comprising:
upper and lower supports, each support having an outer surface configured to engage a vertebra and an inner bearing surface; and
a core comprising upper and lower surfaces configured to engage and articulate with respect to the inner bearing surfaces of the upper and lower supports,
wherein the core is formed as a unitary member with a lateral cut to allow the upper and lower surfaces of the core to move resiliently toward and away from each other without affecting articulation of the upper and lower surfaces of the core with respect to the inner bearing surfaces of the upper and lower supports;
holding the core in a compressed configuration with an instrument; and
inserting the core in the compressed configuration and the supports into the intervertebral space to provide shock absorption.

18. An artificial intervertebral disc comprising:
upper and lower supports, each support having an outer surface configured to engage a vertebra and an inner bearing surface; and
a core comprising upper and lower surfaces configured to engage and articulate with respect to the inner bearing surfaces of the upper and lower supports,
wherein the core is formed as a unitary member with a plurality of lateral cuts to allow the upper and lower surfaces of the core to move resiliently toward and away from each other without affecting articulation of the upper and lower surfaces of the core with respect to the inner bearing surfaces of the upper and lower supports; and
wherein the plurality of lateral cuts have tapering cross sections to provide increasing stiffness with progressive compression.
